(19) United States
(12) Patent Application Publication
(10) Pub. No.: US 2026/0335669 A1
(43) Pub. Date: Oct. 29, 2026

(54) DETACHABLE PHOTOTHERAPY DEVICE

(71) Applicant: Shenzhen Kaiyan Medical Equipment Co., Ltd., Shenzhen (CN)

(72) Inventor: Alain Dijkstra, Amstelveen (NL)

(73) Assignee: Shenzhen Kaiyan Medical Equipment Co., Ltd., Shenzhen (CN)

(21) Appl. No.: 19/738,002

(22) Filed: Jul. 10, 2026

Related U.S. Application Data
(63) Continuation-in-part of application No. 18/334,406, filed on Jun. 14, 2023, now Pat. No. 12,702,858.

(30) Foreign Application Priority Data
Feb. 9, 2023 (CN) 202320160959.6

(51) Int. Cl. A61N 5/06

(52) U.S. Cl. CPC A61N 5/0616; A61N 2005/0652; A61N 2005/0663; A61N 2005/0664

(57) ABSTRACT

A detachable phototherapy device includes a substrate layer having a first surface and a second surface. A conductive layer is disposed on the first surface, the conductive layer comprising a first conductor terminal and a second conductor terminal. A plurality of light sources is electrically coupled to the conductive layer and configured to emit electromagnetic radiation. A first electrode and a second electrode are provided on the second surface, electrically coupled to the first and second conductor terminals, respectively. In some embodiments, a reusable phototherapy module comprising the substrate layer, the conductive layer, the plurality of light sources, and a power source is removably coupled to a replaceable skin-contact patch having a plurality of concave-convex light-transmitting holes aligned with the plurality of light sources. The reusable phototherapy module and the replaceable skin-contact patch may be removably coupled via magnetic attraction.

### CROSS-REFERENCE TO RELATED APPLICATION

This application is a continuation-in-part of U.S. Application No. 18/334,406, titled DETACHABLE PHOTOTHERAPY DEVICE, filed Jun. 14, 2023, which is hereby incorporated by reference in its entirety.

### TECHNICAL FIELD

The present invention relates generally to the field of Photobiomodulation or light therapy, and more specifically to a detachable and reusable phototherapy device.

### BACKGROUND ART

Bandages are applied on wounds to facilitate wound healing. The bandages serve multiple purposes such as keeping severed skin together, applying an antiseptic substance to the wound area, and protecting the wound from environmental elements such as dust, water, and outside micro-organisms such as bacteria and fungi. However, bandages are generally designed for single use and once removed from a wound are thrown away contributing to medical waste generation. Moreover, attachment and removal of the bandages cause some amount of discomfort to a user as the antiseptic portion of the bandage tends to relatively strongly adhere to the skin of the user. Moreover, bandages do little in terms of promoting and expediting wound healing. Therefore, some rather more severe wounds may take a relatively long time and the bandages may need to be replaced several times during that period.

Whereas, electromagnetic radiation, such as visible light is known to have several benefits regarding wound healing. For example, blue light is known to have bactericidal and antiseptic properties. Moreover, red light is known to have replenishing and rejuvenating effects on the skin, as red light promotes the production of collagen and Adenosine Triphosphate (ATP) in skin cells. However, little has been done to combine the properties of electromagnetic radiation with bandages to achieve a combination of benefits available from the bandages and the electromagnetic radiation. Some solutions present in the art combine Organic Light Emitting Diode (OLED) based modules with the bandages to achieve the combined benefits. However, such solutions are complicated in construction and are relatively expensive to design and manufacture.

Therefore, there is a need in the art for phototherapy-based devices that do not suffer from the aforementioned deficiencies.

### OBJECTS OF THE INVENTION

Some of the objects of the invention are as follows:

An object of the present invention is to provide a detachable phototherapy device that can be attached to the skin of a user to enhance wound healing and skin regeneration.

Another object of the invention is to provide a detachable phototherapy device that is detachable and can be reused several times.

Another object of the invention is to provide a detachable phototherapy device that is powered using a battery contained within.

Another object of the invention is to provide a detachable phototherapy device that is adapted to emit blue light for its antiseptic and bactericidal properties and red light for its skin regeneration properties.

### SUMMARY

According to an aspect of the present invention, there is provided a detachable phototherapy device. The detachable phototherapy device includes a substrate layer comprising a first surface oriented towards a first direction, and a second surface oriented towards a second direction opposite to the first direction. A conductive layer is printed on the first surface of the substrate layer, the conductive layer comprising a first conductor terminal and a second conductor terminal. A plurality of light sources is electrically coupled to the conductive layer, the plurality of light sources configured to emit electromagnetic radiation towards the first direction. A first electrode and a second electrode are provided on the second surface, the first electrode electrically coupled to the first conductor terminal, and the second electrode electrically coupled to the second conductor terminal.

In one embodiment, the detachable phototherapy device further includes a battery having a first battery terminal and a second battery terminal, wherein in a use state, the battery is electrically coupled between the first electrode and the second electrode. In one embodiment, the detachable phototherapy device further includes a replaceable skin-contact patch removably coupled to the substrate layer, the replaceable skin-contact patch including an adhesive skin-contact surface and at least one optically transmissive region aligned with the plurality of light sources. In one embodiment, the at least one optically transmissive region comprises a plurality of concave-convex light-transmitting holes. In one embodiment, the substrate layer and the replaceable skin-contact patch are coupled via magnetic attraction. In one embodiment, the detachable phototherapy device further includes a deformable light board having a plurality of slots enabling multi-directional deformation. In one embodiment, the plurality of light sources includes first light sources configured to emit electromagnetic radiation in a red wavelength range and second light sources configured to emit electromagnetic radiation in a blue wavelength range.

According to another aspect of the present invention, there is provided a detachable phototherapy device comprising a reusable phototherapy module and a replaceable skin-contact patch. The reusable phototherapy module comprises a substrate layer having a first surface and a second surface, a conductive layer disposed on the first surface, a plurality of light sources electrically coupled to the conductive layer, and a power source. The replaceable skin-contact patch is removably coupled to the reusable phototherapy module and includes a plurality of concave-convex light-transmitting holes aligned with the plurality of light sources. Each concave-convex light-transmitting hole includes a first opening portion facing the reusable phototherapy module and a second opening portion facing a wound surface.

In one embodiment, the reusable phototherapy module further comprises a first electrode and a second electrode disposed on the second surface of the substrate layer, the first electrode electrically coupled to a first conductor terminal of the conductive layer and the second electrode electrically coupled to a second conductor terminal of the conductive layer. In one embodiment, the first opening portion is convex and the second opening portion is concave. In one embodiment, an inner wall of each concave-convex light-transmitting hole is coated with an antibacterial coating. In one embodiment, the replaceable skin-contact patch includes a skin-contact hydrogel layer and a polyurethane foam layer. In one embodiment, the reusable phototherapy module includes a deformable light board having a plurality of slots enabling multi-directional deformation. In one embodiment, the reusable phototherapy module includes a light-transmitting plate having a concave-convex lens structure forming a double-layer lens structure with the concave-convex light-transmitting holes.

According to another aspect of the present invention, there is provided a detachable phototherapy device comprising a reusable phototherapy module and a replaceable skin-contact patch. The reusable phototherapy module comprises a substrate layer having a first surface and a second surface, a conductive layer disposed on the first surface, a plurality of light sources electrically coupled to the conductive layer, and a power source. The replaceable skin-contact patch includes an adhesive skin-contact surface and at least one optically transmissive region aligned with the plurality of light sources. The reusable phototherapy module includes a plurality of first magnetic elements and the replaceable skin-contact patch includes a plurality of second magnetic elements, the plurality of first magnetic elements and the plurality of second magnetic elements configured to removably magnetically couple the reusable phototherapy module to the replaceable skin-contact patch.

In one embodiment, the reusable phototherapy module further comprises a first electrode and a second electrode disposed on the second surface of the substrate layer, the first electrode electrically coupled to a first conductor terminal of the conductive layer and the second electrode electrically coupled to a second conductor terminal of the conductive layer. In one embodiment, the replaceable skin-contact patch includes an identification element, and the reusable phototherapy module includes a controller configured to detect a type or presence of the replaceable skin-contact patch before activating the plurality of light sources. In one embodiment, the controller prevents activation of the plurality of light sources when the replaceable skin-contact patch is not magnetically coupled to the reusable phototherapy module. In one embodiment, the at least one optically transmissive region comprises a plurality of concave-convex light-transmitting holes having a convex first opening portion and a concave second opening portion. In one embodiment, the plurality of light sources includes first light sources and second light sources configured to emit electromagnetic radiation at different wavelengths, and the reusable phototherapy module includes a controller configured to selectively drive the light sources according to a wound treatment protocol.

### BRIEF DESCRIPTION OF FIGURES

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates an exploded view of a detachable phototherapy device, in accordance with an embodiment of the present invention;

FIG. 1B illustrates a top perspective view of the detachable phototherapy device of FIG. 1A;

FIG. 1C illustrates a bottom view of the detachable phototherapy device of FIG. 1A;

FIG. 2 illustrates a bottom perspective view of the detachable phototherapy device in a use state, in accordance with an embodiment of the present invention;

FIG. 3 illustrates a logical circuit diagram of the detachable phototherapy device in the use state, in accordance with an embodiment of the present invention;

FIG. 4A illustrates an exploded view of the detachable phototherapy device, in accordance with another embodiment of the present invention;

FIG. 4B illustrates a top perspective view of the detachable phototherapy device of FIG. 4A;

FIG. 4C illustrates a bottom view of the detachable phototherapy device of FIG. 4A;

FIG. 5 illustrates a use case scenario of the detachable phototherapy device, with the detachable phototherapy device applied on the skin of a user, in accordance with an embodiment of the present invention; and

FIG. 6 is an alternate embodiment showing the device with a control unit communicating to an external communication device.

FIG. 7 illustrates an exploded view of a reusable phototherapy module and a replaceable skin-contact patch, in accordance with an embodiment of the present invention.

FIG. 8A illustrates a cross-sectional view of a replaceable skin-contact patch with concave hydrogel columns, in accordance with an embodiment of the present invention.

FIG. 8B illustrates a cross-sectional view of a replaceable skin-contact patch with convex hydrogel columns, in accordance with an embodiment of the present invention.

FIG. 9 illustrates a deformable light board with a slot structure and a cross-sectional view, in accordance with an embodiment of the present invention.

FIG. 10 illustrates an assembled view of the detachable phototherapy device with a magnetic-attraction connection, in accordance with an embodiment of the present invention.

FIG. 11 illustrates detailed views of lens elements for a double-layer lens structure, in accordance with an embodiment of the present invention.

FIG. 12 illustrates a cross-sectional view of the detachable phototherapy device showing the reusable phototherapy module coupled to a replaceable skin-contact patch, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

In the context of this specification, terms like “light”, “radiation”, “irradiation”, “emission” and “illumination”, etc. refer to electromagnetic radiation in wavelength ranges varying from the visible light wavelengths (380-700 nm) to Infrared (IR) wavelengths (780 nm-1 mm), wherein the range is inclusive of visible light and IR wavelengths. The IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, “Light Emitting Diodes (LEDs)” are envisaged to be semiconductor devices that emit electromagnetic radiation when current is applied through them. LEDs are characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue and white colors), Surface Mount LEDs, Bi-color LEDs, Pulse Width Modulated RGB (Red-Green-Blue) LEDs, and high-power LEDs, etc.

Materials used in the construction of LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGalnP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights, and copper and zinc doped zinc sulfide-based phosphor to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to Nth Degree Technologies Worldwide Inc., which are included herein by reference, in their entirety. The one or more LEDs, in that regard, may be provided as a printable composition of the micro-LEDs, printed on a substrate.

In the context of the specification, the term “Iontophoresis” refers to application of direct current to the epidermis of a patient. Iontophoresis includes the application of galvanic current on the positive polarity. During an iontophoresis treatment, a product that has an acidic pH and is water-soluble can be used. Gels, serums, or even a mask can be applied all over the area to be treated.

It is envisaged that a detachable phototherapy device be provided that can be used as a bandage for promoting wound healing and skin regeneration. The detachable phototherapy device may include a conductive layer printed and/or deposited on a substrate layer, and several light sources, such as, but not limited to, Light Emitting Diodes (LEDs), electrically coupled to the conductive layer. The substrate layer may be made up of sulphuric acid paper. Further, the substrate layer may include a first portion on which the several LEDS, one of two electrodes, and a battery may be provided. Further, the substrate layer may include a second portion on which a second electrode of the two electrodes may be provided.

The first and the second portion may be connected through a bend portion. The bend portion may be adapted to bend, in a manner that the second portion is moved towards the first portion to position the battery between the two electrodes, thereby completing an electrical power circuit for the several light sources. The battery may be a rechargeable battery. Further, electrodes made from a conductive material, such as conductive silicone, may be attached to the conductive layer to provide iontophoresis to a portion of skin of a user. It is also envisaged that the substrate layer may be detachably attached to a sticker made out of a non-woven fabric material, through adhesive bonding. The sticker with the substrate layer detachably attached therewith and including the several sources powered by the battery, therebetween, may then be applied to a wounded portion of the skin of the user. Referring to the drawings, the present invention will now be discussed in more detail.

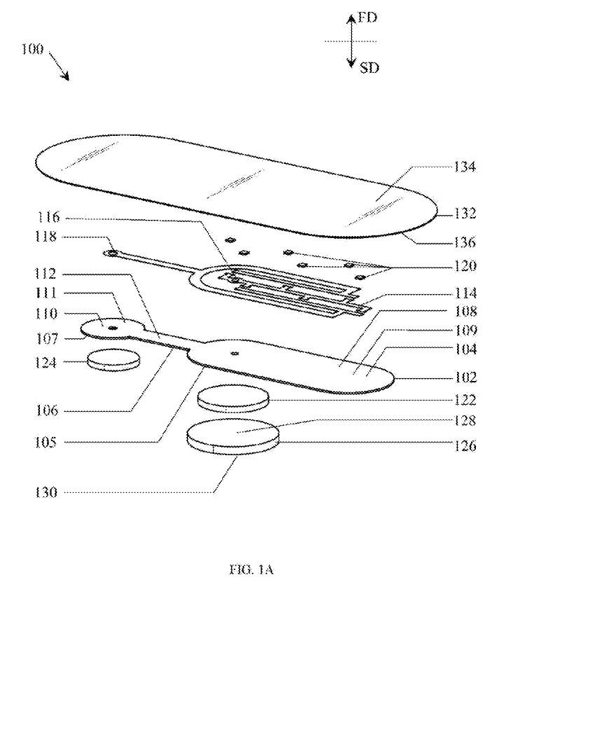

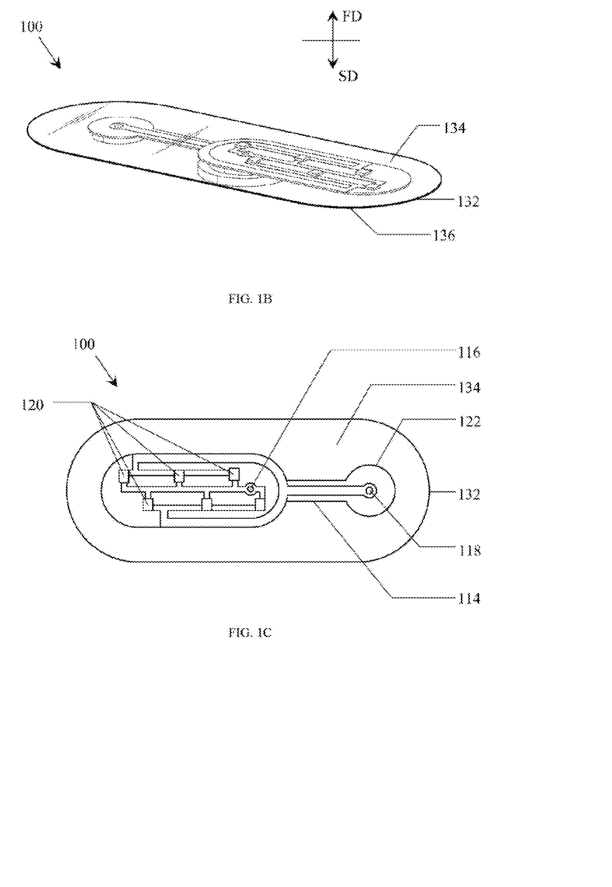

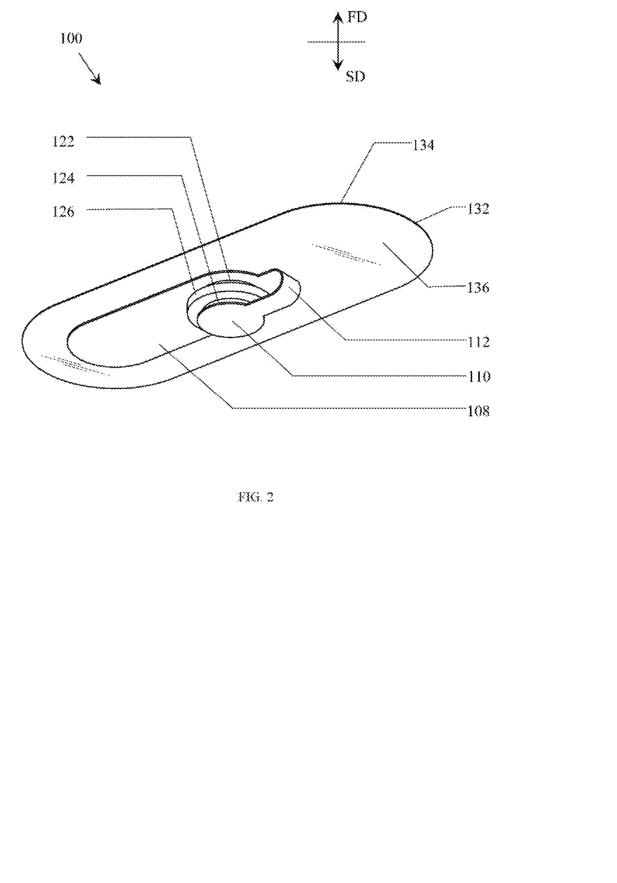

FIG. 1A illustrates an exploded view of a detachable phototherapy device 100 (hereinafter referred to as “the device 100”), in accordance with an embodiment of the present invention. The device 100 includes a substrate layer 102. In several embodiments of the invention, the substrate layer 102 is made up of sulphuric acid paper. The sulphuric acid paper offers several advantages, such as, the sulphuric acid paper is characterized by high density, stability, heat resistance, grease resistance, water resistance, no loose fibers as well as low surface energy, thereby imparting good non-stick or release properties to the substrate layer 102 and also helps in wound healing. The aforementioned properties of the sulphuric acid paper make the sulphuric acid paper ideal for bandages.

The substrate layer 102 includes a first substrate surface 104 oriented towards a first direction (FD). The first direction (FD) may be in a direction of application of the device 100 on a skin of a user. The substrate layer 102 further includes a second substrate surface 106 oriented towards a second direction (SD). The second direction (SD) is envisaged to be opposite to the first direction (FD). Further, the substrate layer 102 includes a first portion 108. In one embodiment of the invention, the first portion 108 is envisaged to be longitudinal in shape, with a first predetermined thickness. The first portion 108 includes a first segment 109 of the first substrate surface 104. Further, the first portion 108 includes a third segment 105 of the second substrate surface 106.

The substrate layer 102 further includes a second portion 110. In one embodiment of the invention, the second portion 110 is envisaged to be circular in shape, with a second predetermined thickness. In several embodiments of the invention, the first predetermined thickness is equal to the second predetermined thickness. The second portion 110 includes a second segment 111 of the first substrate surface 104, and a fourth segment 107 of the second substrate surface 106. A bend portion 112 connects the first portion 108 with the second portion 110. In several embodiments of the invention, the bend portion 112 is rectangular in shape. Moreover, in several embodiments of the invention, a width of the bend portion 112 is smaller than respective widths of the first portion 108 and the second portion 110, making the bend portion 112 more slender and easier to bend with respect to the first portion 108 and the second portion 110.

In some embodiments, the substrate layer 102 may be a single unified layer without separate first and second portions connected by a bend portion. In such embodiments, the substrate layer 102 may include the first substrate surface 104 oriented towards the first direction and the second substrate surface 106 oriented towards the second direction, with the conductive layer 114 disposed on the first substrate surface 104 and the first electrode 122 and the second electrode 124 disposed on the second substrate surface 106.

A conductive layer 114 has been printed (or deposited) on the first substrate surface 104 of the substrate layer 102. The conductive layer 114 may be made up of an electrically conductive material such as, but not limited to, copper, aluminum, etc. The conductive layer 114 may be printed by depositing the electrically conductive material onto the first substrate surface 104 of the substrate layer 102. The conductive layer 114 includes a first conductor terminal 116 and a second conductor terminal 118. In one embodiment of the invention, the first conductor terminal 116 is located on the first segment 109 of the first substrate surface 104, and the second conductor terminal 118 is located on the second segment 111 of the first substrate surface 104.

Further, the device 100 includes a plurality of light sources 120 electrically coupled to the conductive layer 114. In one embodiment of the invention, the plurality of light sources 120 includes a plurality of Light Emitting Diodes (LEDs). In several alternate embodiments, the plurality of light sources 120 may also include incandescent light sources, or lasers, without departing from the scope of the invention. In one embodiment of the invention, all of the plurality of light sources 120 may be provided on the first segment 109 of the first substrate surface 104.

The plurality of light sources 120 are configured to emit electromagnetic radiation towards the first direction (FD). In that regard, the plurality of light sources 120 may be configured to emit the electromagnetic radiation of wavelength ranges including 620-660 nm (red light) and 450-495 nm (blue light). The red light is known to promote skin repair, regeneration and rejuvenation by enhancing the production of collagen and Adenosine Triphosphate (ATP). Further, the blue light is known for its bactericidal and antiseptic properties. Therefore, both the red light and the blue light would expedite the process of wound healing.

Further, the device 100 includes a first electrode 122 and a second electrode 124. The first electrode 122 is coupled to the first portion 108 on the third segment 105 of the second substrate surface 106. The first electrode 122 may be coupled to the first portion 108 using adhesive bonding. The second electrode 124 is coupled to the second portion 110 on the fourth segment 107 of the second substrate surface 106. The second electrode 124 may be coupled to the second portion 110 also using the adhesive bonding. Further, the first electrode 122 is electrically coupled to the first conductor terminal 116. Also, the second electrode 124 is electrically coupled to the second conductor terminal 118. In that regard, the first portion 108 and the second portion 110 may include holes through which electrical conductors may pass, and connect the first electrode 122 with the first conductor terminal 116 and the second electrode 124 with the second conductor terminal 118.

The device 100 further includes a battery 126. The battery 126 includes a first battery terminal 128 and a second battery terminal 130. In one embodiment of the invention, the first battery terminal 128 may be a positive terminal and the second battery terminal 130 may be a negative terminal. In an alternate embodiment, the first battery terminal 128 may be the negative terminal and the second battery terminal 130 may be the positive terminal. In one embodiment of the invention, in a non-use state of the device 100, the battery 126 is electrically coupled to the first electrode 122 through the first battery terminal 128, and the second battery terminal 130 is oriented towards the second direction (SD). In another embodiment of the invention, in the non-use state of the device 100, the battery 126 may be electrically coupled to the second electrode 124 through the second battery terminal 130, and the first battery terminal 128 may be oriented towards the second direction (SD).

In addition to the electrical coupling, the battery 126 may also be mechanically coupled to either one of the substrate layer 102, the first electrode 122, and the second electrode 124. The mechanical coupling with the substrate layer 102 may be achieved by attaching the battery 126 adhesively to one of the first substrate surface 104 and the second substrate surface 106. The mechanical coupling with the first electrode 122 or the second electrode 124 may be achieved through snap-fit fastening, clip-based fastening, or magnetic fastening. In that regard, in several embodiments of the invention, the first electrode 122 and the second electrode 124 may be permanent magnets. In several embodiments of the invention, the battery 126 may be a rechargeable battery. For example, the battery 126 may be a Nickel-Metal-Hydride battery, Lithium-ion battery, or a Lithium polymer battery.

In some embodiments, in a use state of the device 100, the battery 126 may be electrically coupled between the first electrode 122 and the second electrode 124, with the first battery terminal 128 electrically coupled to the first electrode 122 and the second battery terminal 130 electrically coupled to the second electrode 124. The battery 126 may be coupled between the electrodes through bending of the substrate layer 102 at the bend portion 112, through direct placement of the battery 126 in contact with both electrodes, through a battery holder or clip mechanism, or through magnetic attachment where the electrodes are permanent magnets.

In several embodiments of the invention, the device 100 further includes a sticker 132. The sticker 132 further includes a first sticker surface 134 oriented towards the first direction (FD), and a second sticker surface 136 oriented towards the second direction (SD). The first sticker surface 134 and the second sticker surface 136 may be provided with adhesive layers. In several embodiments of the invention, the adhesives provided on the first sticker surface 134 and the second sticker surface 136 may be identical. In several alternate embodiments, the adhesives provided on the first sticker surface 134 and the second sticker surface 136 may be non-identical. The adhesive layer on the first sticker surface 134 may be provided with a liner strip over the adhesive layer, in the non-use state of the device 100. The adhesive layer of the second sticker surface 136 may be used to bond the first substrate surface 104. The conductive layer 114 and the plurality of light sources 120 may be disposed between the substrate layer 102 and the sticker 132.

In other words, the substrate layer 102 is adapted to be detachably attached to the second sticker surface 136 through adhesive bonding, such that the conductive layer 114 and the plurality of light sources 120 are sandwiched between the first substrate surface 104 of the substrate layer 102 and the second sticker surface 136 of the sticker 132. Further, the sticker 132 is adapted to be detachably attached to a body portion of a user, along the first sticker surface 134. The sticker 134 may be attached to the body portion of the user, after removal of the liner strip from over the adhesive layer of the first sticker surface 134. In several embodiments of the invention, the sticker 132 may be made up of a non-woven fabric material. The non-woven fabric materials are characterized by their ability to absorb moisture, resist bacterial infestation, and repel liquids, resilience, softness, sterility, strength, cushioning, thermal insulation, and washability.

FIG. 1B illustrates a top perspective view of the device 100 of FIG. 1A. FIG. 1C illustrates a bottom view of the device 100 of FIG. 1A. FIGS. 1B and 1C illustrate the sticker 132 with the first sticker surface 134 and the second sticker surface 136. Further illustrated are the conductive layer 114, the plurality of light sources 120, the first conductor terminal 116, the second conductor terminal 118, and the first electrode 122.

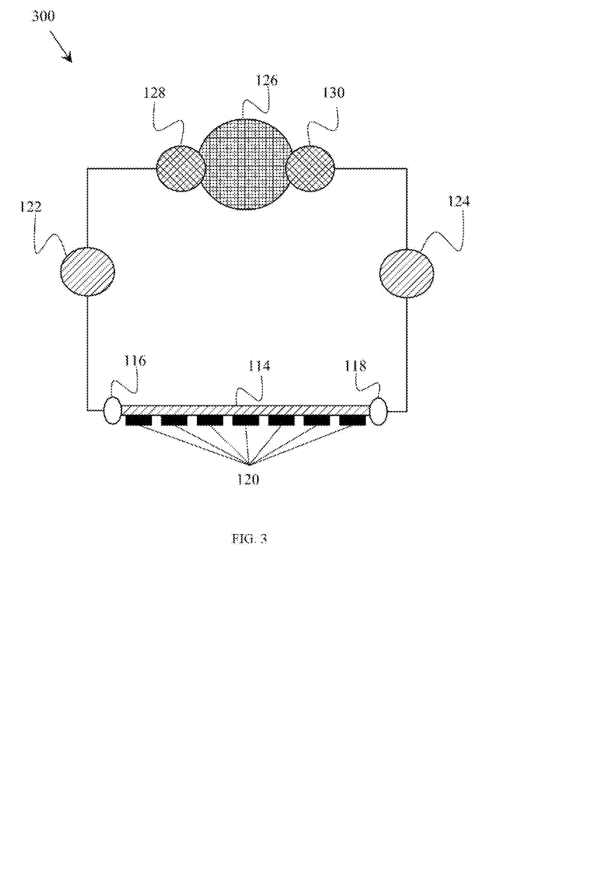

FIG. 2 illustrates a bottom perspective view of the device 100 in a use state, in accordance with an embodiment of the present invention. In the use state, the substrate layer 102 is adapted to bend at the bend portion 112, allowing the battery 126 to be electrically coupled between the first electrode 122 and the second electrode 124. In that regard, the first battery terminal 128 is electrically coupled to the first electrode 122, and the second battery terminal 130 is electrically coupled to the second electrode 124. As a result, a conductive circuit including the conductive layer 114, the plurality of light sources 120, the first electrode 122, the second electrode 124, and the battery 126 is completed.

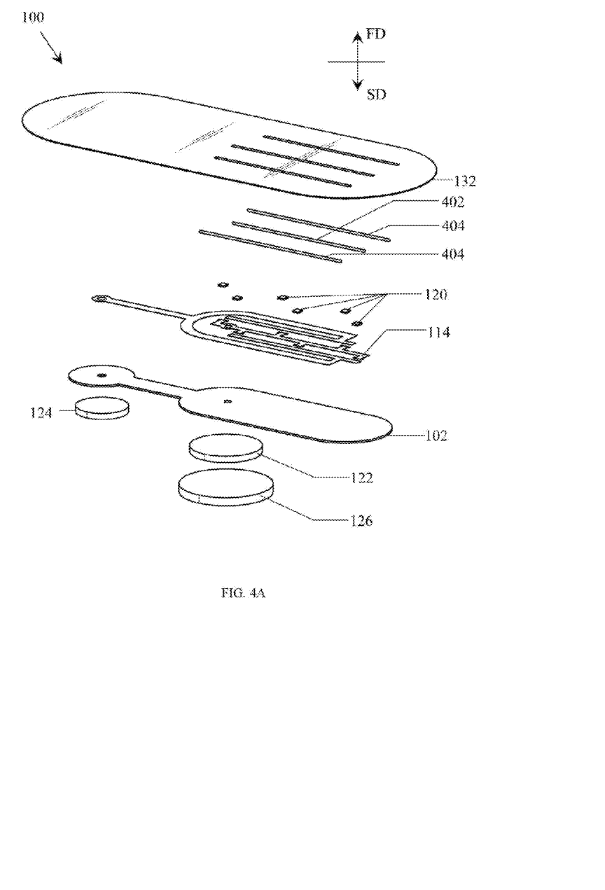

FIG. 3 illustrates a logical circuit diagram 300 of the device 100 in the use state, in accordance with an embodiment of the present invention. As illustrated in FIG. 3 , the battery 126 includes the first battery terminal 128 and the second battery terminal 130. The first battery terminal 128 is electrically coupled to the first electrode 122, and the second battery terminal 130 is electrically coupled to the second electrode 124. The plurality of light sources 120 have been electrically attached to the conductive layer 114. The conductive layer 114 includes the first conductor terminal 116 and the second conductor terminal 118. The first conductor terminal 116 is electrically coupled to the first electrode 122, and the second conductor terminal 118 is electrically coupled to the second electrode 124. As a result, a circuit is completed allowing the battery 126 to power the plurality of light sources 120.

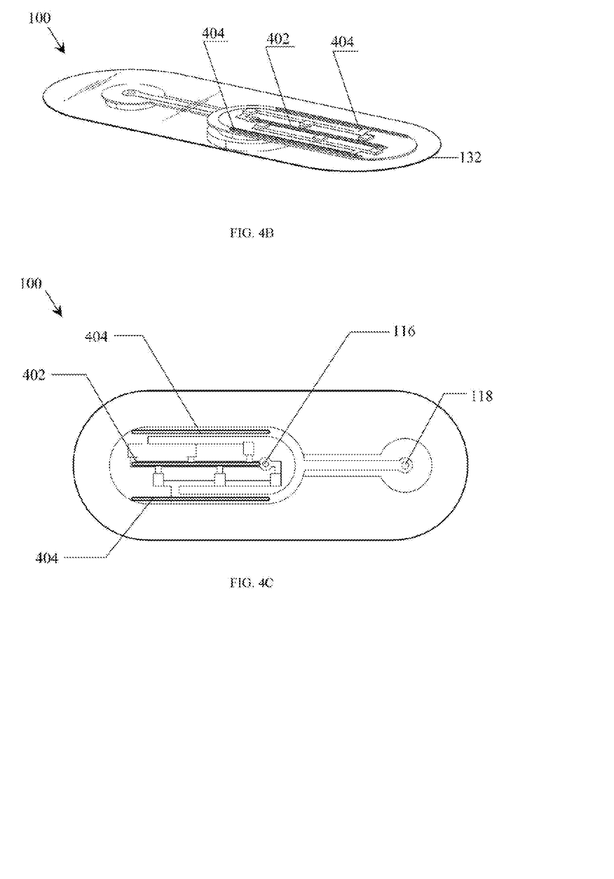

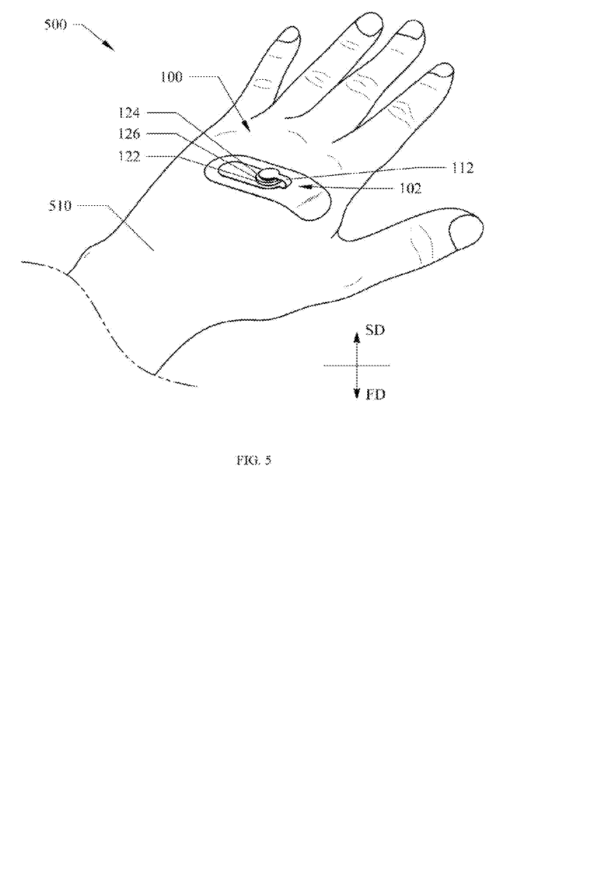

FIG. 4A illustrates an exploded view of the detachable phototherapy device 100, in accordance with another embodiment of the present invention. FIG. 4B illustrates a top perspective view of the detachable phototherapy device 100 of FIG. 4A. FIG. 4C illustrates a bottom view of the detachable phototherapy device 100 of FIG. 4A. Referring to FIGS. 4A, 4B, and 4C, the detachable phototherapy device 100 further includes at least one first electrode 402 attached to the conductive layer 114 and electrically coupled to the first conductor terminal 116. Further, the detachable phototherapy device 100 includes at least one second electrode 404 attached to the conductive layer 114 and electrically coupled to the second conductor terminal 118. The at least one first electrode 402 and the at least one second electrode 404 are envisaged to be made up of electrically conductive materials, so that they are able to be charged as per the polarity of the first conductor terminal 116 and the second conductor terminal 118, respectively. In that regard, one of the at least one first electrode 402 and the at least one second electrode 404 will be positively charged and the other would be negatively charged. In several embodiments of the invention, the at least one first electrode 402 and the at least one second electrode 404 are made up of electrically conductive silicone material. The electrically conductive silicone material is typically made from impregnating silicone material with carbon. As a consequence, the at least one first electrode 402 and the at least one second electrode 404 are configured to provide iontophoresis therapy to a portion of skin of a user.

Iontophoresis involves active transdermal drug delivery involving delivery of drug ions through the skin using low level electric current. When Direct Current (DC) is applied to an ionized drug solution, ions that have same charge as the current are repelled by the current and delivered through the skin. For example, the ionized drug solution may be applied to a hand of the user and the device 100 may be attached to the area where the ionized drug solution has been applied. The DC current is applied through the conductive layer 114 and the at least one first electrode 402 and the at least one second electrode 404. The ions in the drug solution are repelled and absorbed through the hand of the user and provide pain relief to the user. Iontophoresis can be used with water soluble ionic medications to treat acute tendonitis, pain associated with calcific deposits, and provide dermal anesthesia. Iontophoresis has several advantages over alternatives to injections and oral medications. Iontophoresis is virtually painless and is non-invasive minimizing the risk of infection and tissue necrosis and tendon rupture.

FIG. 5 illustrates a use case scenario 500 of the device 100, with the device 100 applied on the skin of a user 510, in accordance with an embodiment of the present invention. In the use case scenario, the liner strip on the adhesive layer of the first sticker surface 134 may be removed, and the first sticker surface 134 may be adhesively applied to an affected area on the skin of the user 510. The substrate layer 102 may be bent at the bend portion 112 electrically coupling the battery 126 between the first electrode 122 and the second electrode 124. The circuit may therefore be completed and the plurality of light sources 120 would begin to emit the electromagnetic radiation in red and blue visible light frequencies to provide bactericidal and anti-septic effect and promote skin regeneration and repair in the affected area of the skin of the user. Further, in several embodiments, the at least one first electrode 402 and the at least one second electrode 404 would be able to provide iontophoresis therapy to a portion of the skin of the user 510, where the device 100 has been applied. After use, the substrate layer 102 with the conductive layer 114, the plurality of light sources 120, the first electrode 122, the second electrode 124, and the battery 126 may be removed from the sticker 132 and adhesive bonded to another new sticker. This allows the device 100 to be used several times over with only the sticker 132 having to be discarded.

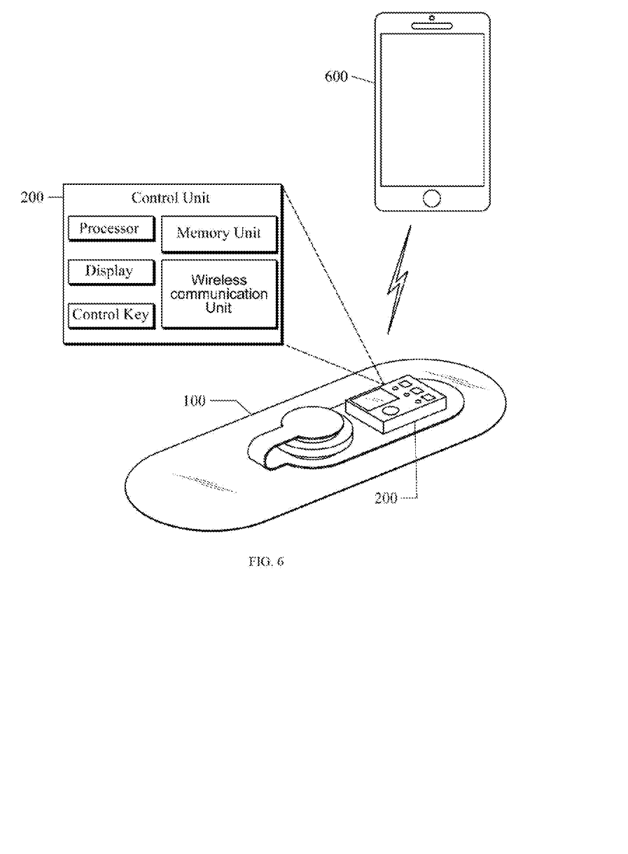

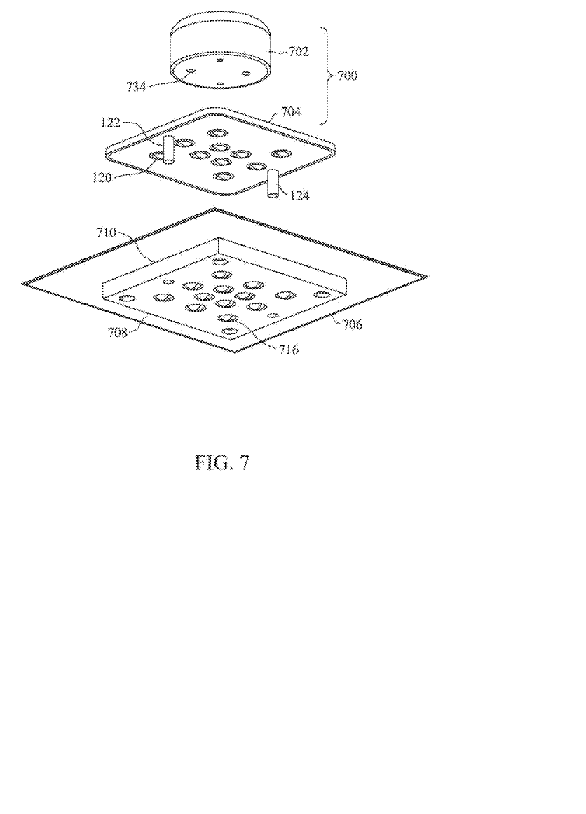

FIG. 6 shows an alternative embodiment, the device 100 includes a control unit 200 configured to connect with an external communication device 600 and receive an input from the external communication device 600. The control unit includes a processor, a memory unit control keys and a display. The memory unit includes machine-readable instructions for the processor to execute. The communication with the external communication device 600 is routed through the display and control keys via wireless communication unit. The control unit 200 is configured to connect with the external communication device 600 and receive an input. The device 100 allows a user to provide input through the control keys and the display unit to switching between different therapies, such as TENS, EMS, iontophoresis, switching between continuous and pulse mode of operation of the LEDs or modification of emission characteristics of the plurality of LEDs such as wavelength of the irradiation, setting time duration of different therapies using a timer.

The invention as described above offers several advantages. For instance, the detachable phototherapy device is very simple in design and construction. Further, the detachable phototherapy device uses commonly available materials. The simplicity in design and construction, and the use of commonly available materials allow the detachable phototherapy device to be mass-produced with minimal capital expenditure, and to be made available in the market at significantly lower prices. Also, the detachable phototherapy device can be reused several times and is, therefore, cost-effective for the end user and minimizes waste generation.

In some embodiments, the substrate layer 102 with the conductive layer 114, the plurality of light sources 120, and the first electrode 122 and second electrode 124 is removably coupled to a replaceable skin-contact patch. The replaceable skin-contact patch includes an adhesive skin-contact surface and at least one optically transmissive region aligned with the plurality of light sources 120. In such embodiments, the substrate layer 102 is detached from the replaceable skin-contact patch after use and coupled to a new replaceable skin-contact patch, allowing the substrate layer 102 and its components to be reused.

In some embodiments, the replaceable skin-contact patch used with the substrate layer 102 and its components as described in connection with FIGS. 1A-6 includes any of the dressing features described herein, including those described in more detail below with reference to FIGS. 7-11. For example, the replaceable skin-contact patch includes a plurality of concave-convex light-transmitting holes 712 aligned with the plurality of light sources 120, each concave-convex light-transmitting hole 712 having a convex first opening portion 714 facing the substrate layer 102 and a concave second opening portion 716 facing a wound surface. In an embodiment, the replaceable skin-contact patch includes concave-shaped hydrogel elements 718 or convex-shaped hydrogel elements 720 seated within the concave-convex light-transmitting holes 712. In an embodiment, an inner wall of each concave-convex light-transmitting hole 712 is coated with an antibacterial coating 726, such as a nano-silver coating, a chitosan coating, a zinc oxide coating, a copper-based coating, or an antimicrobial hydrogel coating. In an embodiment, the replaceable skin-contact patch includes a multi-layer dressing composition including a skin-contact hydrogel layer 724 and a polyurethane foam layer 722. In an embodiment, the replaceable skin-contact patch includes micropores loaded with growth-promoting factors such as epidermal growth factor (EGF) and wound healing-promoting drugs. In an embodiment, the growth-promoting factors and wound healing-promoting drugs are provided as a surface coating applied to the hydrogel layer or hydrogel lens surfaces, or the dressing material is impregnated with medication by soaking the dressing in medication solutions. In an embodiment, the substrate layer 102 and the replaceable skin-contact patch are coupled via magnetic attraction, wherein the substrate layer 102 includes a plurality of first magnetic elements 734 and the replaceable skin-contact patch includes a plurality of second magnetic elements 736 configured to removably magnetically couple the substrate layer 102 to the replaceable skin-contact patch. In an embodiment, a deformable light board 728 having a plurality of slots 730 enabling multi-directional deformation is provided with the substrate layer 102 to enable the device 100 to conform to curved body surfaces. In an embodiment, a light-transmitting plate 732 having a concave-convex lens structure is provided with the substrate layer 102, and the light-transmitting plate 732 and the concave-convex light-transmitting holes 712 of the replaceable skin-contact patch form a double-layer lens structure for directional light conduction. In an embodiment, the replaceable skin-contact patch includes an identification element, such as an RFID tag, an NFC tag, a resistor code, a QR code, or a conductive bridge, and the device 100 is configured to detect a type or presence of the replaceable skin-contact patch before activating the plurality of light sources 120. In an embodiment, the replaceable skin-contact patch includes conductive electrode contact regions aligned with the first electrode 122 and the second electrode 124 of the substrate layer 102, enabling microcurrent therapy or iontophoresis therapy to be delivered through the replaceable skin-contact patch to the wound surface. In an embodiment, the replaceable skin-contact patch is provided as any of three types of replaceable dressings: a polyurethane perforated type with embedded hydrogel lens cylinders, growth-promoting factors, wound healing-promoting drugs, and an antibacterial coating; a full hydrogel type without perforations having stretchable properties and a wound-approximating effect; or an ultra-thin silicone type for superficial wounds. In an embodiment, the replaceable skin-contact patch includes a dynamically adaptive dressing layer having a multi-layer composite structure including a directionally contractile thermally responsive hydrogel layer (DCTH), a light-responsive antibacterial layer with embedded photothermal nanozyme (PNZ) particles, and a medical-grade polyethylene glycol (PEG) hydrogel layer loaded with epidermal growth factor (EGF). It is to be understood that the dressing features described herein in connection with the reusable phototherapy module 700 and FIGS. 7-11 are not limited to use with the reusable phototherapy module 700 and are also used with the substrate layer 102 and its components as described in connection with FIGS. 1A-6.

In accordance with further embodiments of the present invention, the detachable phototherapy device 100 is configured as a system comprising a reusable phototherapy module 700 and a replaceable skin-contact patch 706 that is removably coupled to the reusable phototherapy module 700. In such embodiments, the reusable phototherapy module 700 includes the substrate layer 102, the conductive layer 114, the plurality of light sources 120, the first electrode 122, the second electrode 124, the battery 126, and a controller. The reusable phototherapy module 700 is configured to be attached to and detached from the replaceable skin-contact patch 706. The replaceable skin-contact patch 706 includes an adhesive skin-contact surface 708 and at least one optically transmissive region 710 aligned with the plurality of light sources 120. After use, the reusable phototherapy module 700 is detached from the replaceable skin-contact patch 706 and coupled to a new replaceable skin-contact patch 706. In an embodiment, the reusable phototherapy module 700 is reused for 50 or more treatment sessions, while the replaceable skin-contact patch 706 is replaced after each use. FIG. 7 illustrates an exploded view of such an embodiment.

Referring to FIG. 7, the detachable phototherapy device 100 includes three main components stacked vertically. At the top is a detachable power supply unit 702 having a cylindrical housing. The detachable power supply unit 702 includes a magnetic-attraction interface 756 on its bottom surface for removable connection to the phototherapy light source module 704 below. The detachable power supply unit 702 includes a built-in rechargeable lithium battery 126, for example, a 200 mAh battery, supporting Type-C fast charging. In an embodiment, the detachable power supply unit 702 is fully charged in approximately 40 minutes. The detachable power supply unit 702 further includes a power indicator and a low-power management chip for a stable power supply under a waterproof and sealed structure. In an embodiment, the detachable power supply unit 702 is detached separately from the phototherapy light source module 704 for independent charging.

In the middle of the assembly shown in FIG. 7 is a phototherapy light source module 704. The phototherapy light source module 704 includes the substrate layer 102 with the conductive layer 114 disposed thereon, and an array of light sources, for example, 1 to 12 LEDs in a 3030 package. The first electrode 122 and the second electrode 124 are disposed on the second substrate surface 106 and are electrically coupled to the first conductor terminal 116 and the second conductor terminal 118, respectively. The phototherapy light source module 704 further includes a controller and a display showing treatment time and operational parameters. In an embodiment, the phototherapy light source module 704 is housed in a food-grade silicone housing or a medical-grade ABS housing. The phototherapy light source module 704 includes built-in electrode contacts that supply power to the light sources after connection with the detachable power supply unit 702.

At the bottom of the assembly shown in FIG. 7 is the replaceable skin-contact patch 706. The replaceable skin-contact patch 706 includes an adhesive skin-contact surface 708 on its bottom for application to the skin of a user. The replaceable skin-contact patch 706 further includes optically transmissive regions 710, which are holes or transparent windows, aligned with the plurality of light sources 120 of the phototherapy light source module 704. In an embodiment, the replaceable skin-contact patch 706 includes a medical-grade pressure-sensitive silicone adhesive at its edges for secure attachment to the skin. The replaceable skin-contact patch 706 is a disposable component configured to be discarded after a single use. The reusable phototherapy module 700, comprising the detachable power supply unit 702 and the phototherapy light source module 704, is configured to be detached from the replaceable skin-contact patch 706 after use and coupled to a new replaceable skin-contact patch 706.

In an embodiment, the replaceable skin-contact patch 706 includes conductive electrode contact regions aligned with the first electrode 122 and the second electrode 124 of the reusable phototherapy module 700. The conductive electrode contact regions enable electrical coupling between the electrodes of the reusable phototherapy module 700 and the skin of a user through the replaceable skin-contact patch 706, thereby enabling microcurrent therapy or iontophoresis therapy to be delivered through the patch to the wound surface. The conductive electrode contact regions are formed of electrically conductive hydrogel, conductive silicone, or other biocompatible conductive materials.

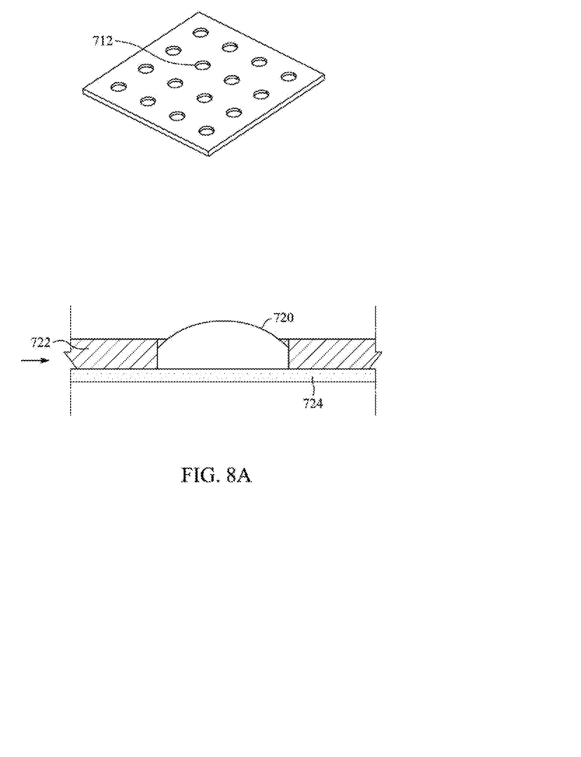

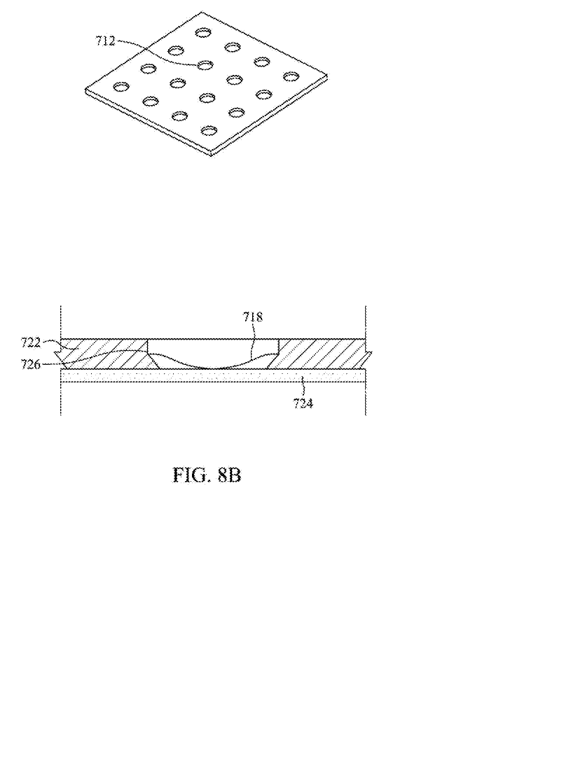

In an embodiment, the replaceable skin-contact patch 706 includes a plurality of concave-convex light-transmitting holes 712 aligned with the plurality of light sources 120. Each concave-convex light-transmitting hole 712 includes a convex first opening portion 714 facing the reusable phototherapy module 700 and a concave second opening portion 716 facing a wound surface. The convex first opening portion 714 is convex, and the concave second opening portion 716 is concave. The convex first opening portion 714 helps collect and concentrate light from the plurality of light sources 120, while the concave second opening portion 716 spreads and diffuses light over the wound surface. This concave-convex hole design reduces light scattering and improves effective light transmission to the wound surface. In an embodiment, the effective light transmittance is raised to over 90%, compared with traditional flat light-transmitting holes. In an embodiment, the concave-convex hole design improves light efficiency by approximately 50% compared to flat holes. Each concave-convex light-transmitting hole 712 is aligned with a corresponding light source position on the phototherapy light source module 704. FIGS. 8A and 8B illustrate cross-sectional views of concave and convex hydrogel column replaceable skin-contact patches, respectively.

Referring to FIG. 8A, a cross-sectional view of a replaceable skin-contact patch with concave hydrogel columns, is illustrated. The replaceable skin-contact patch includes a polyurethane foam layer 722 at the top. Seated inside each hole of the polyurethane foam layer 722 is a concave-shaped hydrogel element 718. The concave hydrogel element 718 has a concave, or bowl-shaped, surface facing downward toward the wound surface. A hydrogel adhesive or skin-contact hydrogel layer 724 is provided at the bottom of the replaceable skin-contact patch for skin-friendly contact with the wound surface. The concave surface of the hydrogel element 718 spreads and diffuses light received from the plurality of light sources 120 over a wider area of the wound surface. In an embodiment, the replaceable skin-contact patch of FIG. 8A further includes a drug-loaded polyurethane foam dressing option, wherein the dressing is soaked in medication, or the micropores in the hydrogel elements 718 are loaded with growth-promoting factors such as epidermal growth factor (EGF) and wound healing-promoting drugs.

Referring to FIG. 8B, a cross-sectional view of a replaceable skin-contact patch with convex hydrogel columns, is illustrated. The replaceable skin-contact patch includes a polyurethane foam layer 722 at the top. Seated inside each hole of the polyurethane foam layer 722 is a convex-shaped hydrogel element 720. The convex hydrogel element 720 has a convex, or dome-shaped, surface protruding upward toward the reusable phototherapy module 700. A hydrogel adhesive or skin-contact hydrogel layer 724 is provided at the bottom of the replaceable skin-contact patch. The convex surface of the hydrogel element 720 collects and concentrates light from the plurality of light sources 120 before the light passes through the dressing toward the wound surface. In an embodiment, the dressing holes are various shapes, including circular, strip-shaped, rhombic, or pagoda-shaped, with each hole shape matched with a corresponding hydrogel lens type to enhance light conductivity and adapt to wounds of different shapes.

In an embodiment, an inner wall of each concave-convex light-transmitting hole 712 is coated with an antibacterial coating 726. The antibacterial coating 726 is selected from a nano-silver coating, a chitosan coating, a zinc oxide coating, a copper-based coating, or an antimicrobial hydrogel coating. In an embodiment, the antibacterial coating 726 achieves an antibacterial rate of 99.9% or greater against bacteria such as Escherichia coli and Staphylococcus aureus. The combination of phototherapy with the antibacterial coating 726 provides a synergistic effect for wound treatment.

In an embodiment, the replaceable skin-contact patch 706 includes a multi-layer dressing composition. The dressing includes a skin-contact hydrogel layer 724 and a polyurethane foam layer 722. The skin-contact hydrogel layer 724 provides skin-friendly contact with the wound surface, and the polyurethane foam layer 722 provides moisture absorption and breathability. In an embodiment, the dressing includes a three-layer composite structure comprising a polyurethane base layer, a hydrogel optical layer, and a medical adhesive layer. The medical adhesive layer is a pressure-sensitive adhesive layer using medical-grade silicone adhesive provided at the edge of the dressing.

In an embodiment, the replaceable skin-contact patch 706 includes micropores loaded with growth-promoting factors. The growth-promoting factors include epidermal growth factor (EGF) and wound healing-promoting drugs. The micropores are provided in the hydrogel lenses or in the dressing material itself. The growth-promoting factors are released upon contact with the wound surface to promote wound healing. In an embodiment, the growth-promoting factors and wound healing-promoting drugs are also provided as a surface coating applied to the hydrogel layer or the hydrogel lens surfaces. In an embodiment, the dressing material is impregnated with medication by soaking the dressing in medication solutions. The surface coating includes an EGF coating, a collagen-promoting coating, or an anti-inflammatory drug coating, which is distinct from the antibacterial coating 726.

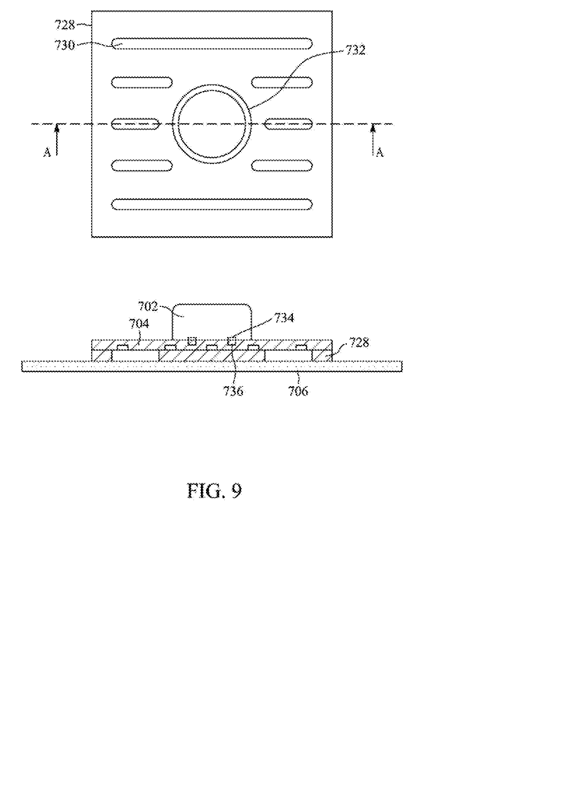

In an embodiment, the reusable phototherapy module 700 includes a deformable light board 728 having a plurality of slots 730 enabling multi-directional deformation. The slots 730 are non-continuous, elongated slots that allow the deformable light board 728 to conform to curved body surfaces such as joints and trunks. In an embodiment, the slots 730 are V-shaped or angled cuts extending across the width of the deformable light board 728. The slots 730 replace traditional flexible hinge designs to enable multi-dimensional bending. The light board main body is made of food-grade silicone housing and is ultra-thin to maximize flexibility. FIG. 9 illustrates a deformable light board 728 with a slot structure.

Referring to FIG. 9, a top plan view of the deformable light board 728 is illustrated. The deformable light board 728 is square or rectangular in shape and includes a grid array of circular light source positions. Multiple parallel elongated slots 730, which are non-continuous long slots, are cut across the surface of the deformable light board 728. The slots 730 enable the deformable light board 728 to flex and conform to curved body surfaces. In an embodiment, a red light source module of the deformable light board 728 adopts a potting and sealing process, achieving an IPX6 waterproof rating. A front light-transmitting plate 732 of the deformable light board 728 integrates an array of independent convex lenses, with each light source corresponding one-to-one with a lens for directional focusing.

FIG. 9 further illustrates an A-A cross-section view showing a layered assembly from top to bottom. At the top is the magnetic-attraction power supply. Below the magnetic-attraction power supply is the phototherapy light source module 704, which is a circuit board with LEDs mounted thereon. Below the phototherapy light source module 704 is the deformable light board 728, which is the slotted flexible substrate. Below the deformable light board 728 is the replaceable skin-contact patch 706. In an embodiment, the flexible substrate is made of polyimide (PI) material with a thickness of 0.1 to 0.3 mm. The surface of the flexible substrate has micron-scale light guide slots distributed radially, with slot walls coated with a TiO₂ anti-reflection coating for directional light conduction. The deformable structure allows the reusable phototherapy module 700 to maintain close contact with the wound surface even on irregular body contours such as joints, trunks, and other curved body surfaces. In an embodiment, the light board and the replaceable skin-contact patch 706 are integrated as a one-piece disposable phototherapy patch, or are provided as separate, independent, detachable components.

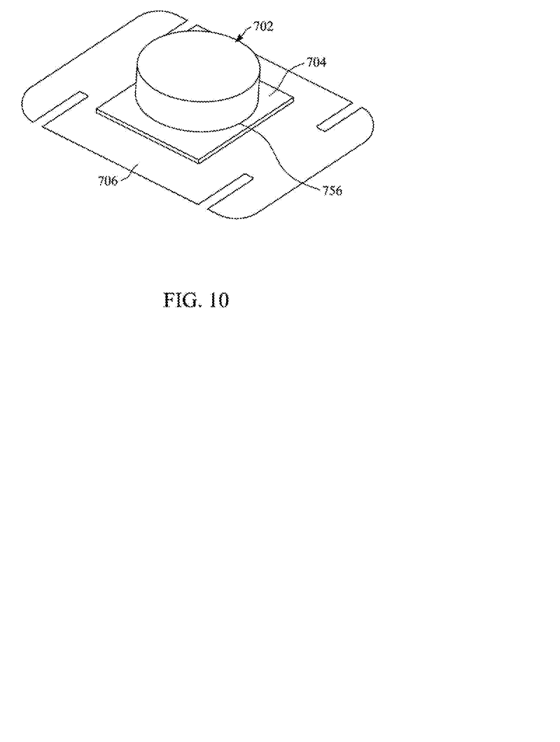

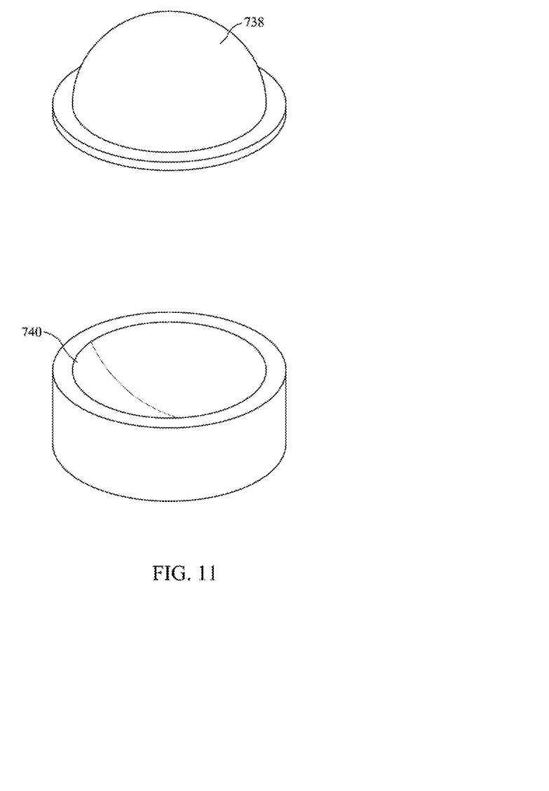

In an embodiment, the reusable phototherapy module 700 and the replaceable skin-contact patch 706 are coupled via magnetic attraction. The reusable phototherapy module 700 includes a plurality of first magnetic elements 734, and the replaceable skin-contact patch 706 includes a plurality of second magnetic elements 736. The plurality of first magnetic elements 734 and the plurality of second magnetic elements 736 are configured to removably magnetically couple the reusable phototherapy module 700 to the replaceable skin-contact patch 706. In an embodiment, flexible magnetic sheets are built into the edges of the replaceable skin-contact patch 706 for precise alignment with corresponding magnetic points on the bottom of the reusable phototherapy module 700. The magnetic-attraction coupling employs multi-point flexible magnetic adsorption, which replaces mechanical buckles and enables quick positioning and replacement of the dressing within approximately 10 seconds. The magnetic-attraction coupling further enables precise alignment between the plurality of light sources 120 and the concave-convex light-transmitting holes 712 of the replaceable skin-contact patch 706. FIG. 10 illustrates an assembled view showing the magnetic-attraction connection.

Referring to FIG. 10, an isometric view of the fully assembled detachable phototherapy device is illustrated. The assembled device includes a rectangular or square base plate, which is the replaceable skin-contact patch 706, with a raised module on top, which is the reusable phototherapy module 700. In an embodiment, edges of the flexible substrate of the reusable phototherapy module 700 include magnetic-attraction interfaces 756 with built-in waterproof sealing rings. When the reusable phototherapy module 700 is magnetically connected to the replaceable skin-contact patch 706, the sealing rating achieves IPX7. In an embodiment, a Hall sensor is built into a magnetic base of the reusable phototherapy module 700 to automatically turn on a circuit when the reusable phototherapy module 700 is properly attached to the replaceable skin-contact patch 706. In an embodiment, a disposable isolation film is provided at the magnetic connection interface. The disposable isolation film is made of polyolefin breathable material with an antibacterial coating on its surface. The disposable isolation film automatically peels off after disassembly to prevent cross-contamination between uses.

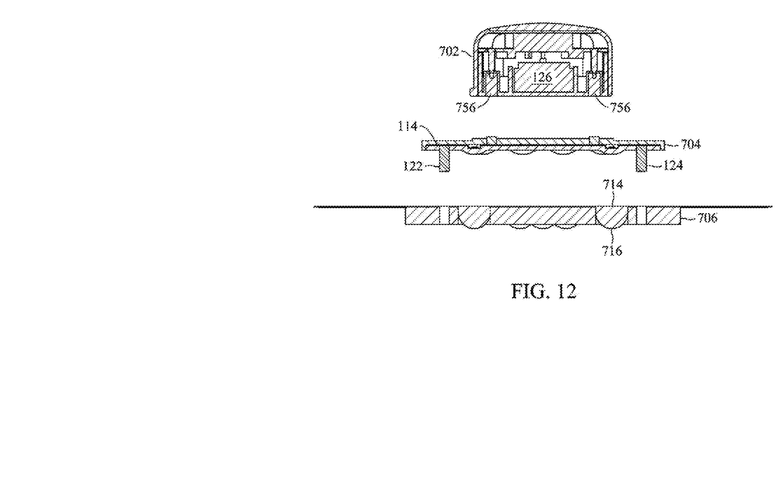

In an embodiment, the reusable phototherapy module 700 includes a light-transmitting plate 732 having a concave-convex lens structure. The light-transmitting plate 732 and the concave-convex light-transmitting holes 712 of the replaceable skin-contact patch 706 form a double-layer lens structure. Each light source is independently matched with a detachable hydrogel lens. The double-layer lens structure provides directional light conduction, with the light-transmitting plate 732 on the reusable phototherapy module 700 concentrating light from each light source and the concave-convex light-transmitting holes 712 in the replaceable skin-contact patch 706 further shaping the light toward the wound surface. In an embodiment, the double-layer lens structure improves light guide efficiency by 40% or more over traditional designs. FIG. 11 illustrates detailed views of lens elements for the double-layer lens structure.

Referring to FIG. 11, two detailed views of individual lens elements are illustrated at an enlarged scale. A first view shows a convex hydrogel lens element 738 having a dome-shaped convex upper portion, which serves as the lens, and a circular base with a flange or rim. The convex hydrogel lens element 738 is designed to be embedded in the dressing layer and aligns with a corresponding LED chip of the plurality of light sources 120. A second view shows a cylindrical hydrogel lens element 740 having a small cylindrical body with a uniform diameter and a slightly recessed or concave top surface. The cylindrical hydrogel lens element 740 represents a concave lens type. The hydrogel lenses are made of biocompatible hydrogel material. In an embodiment, each lamp bead of the plurality of light sources 120 is independently matched with a detachable hydrogel lens to improve the tissue penetration efficiency of light energy.

Referring to FIG. 12, a cross-sectional view of the detachable phototherapy device is illustrated, showing the reusable phototherapy module coupled to a replaceable skin-contact patch 706. At the top of the assembly is a power supply unit 702 having a controller and a battery 126. The controller and the battery 126 are housed within the detachable power supply unit 702 having a rounded upper profile. Magnetic-attraction interfaces 756 are provided on either side of the lower portion of the detachable power supply unit 702 for removable connection to the components below. Beneath the detachable power supply unit 702 is the phototherapy light source module 704, having the substrate layer which includes the conductive layer 114 disposed on the first substrate surface 104. The conductive layer 114 extends across the substrate layer. The first electrode 122 and the second electrode 124 are disposed on the second substrate surface 106 of the substrate layer, positioned on opposite sides. Below the substrate layer is the replaceable skin-contact patch 706, which is configured to contact a wound surface during use. The cross-sectional view of FIG. 12 illustrates the layered arrangement of the components and the spatial relationship between the magnetic-attraction interfaces 756, the first electrode 122 and the second electrode 124, the conductive layer 114, and the substrate layer 102.

In an embodiment, the plurality of light sources 120 includes LED light sources and laser light sources. The LED light sources include 6 to 12 LED chips and emit electromagnetic radiation at wavelengths of 620-660 nm (red light) for promoting collagen synthesis and 450-470 nm (blue light) for providing an antibacterial effect. The laser light sources include 2 to 4 near-infrared laser chips emitting electromagnetic radiation at wavelengths of 808-980 nm (near-infrared laser) with a penetration depth of 5 to 10 mm for deep inflammation relief. In an embodiment, the plurality of light sources 120 provides an optical power density of 30 mW/cm². The different wavelength light sources are independently controlled. In an embodiment, the controller is configured to adjust the power of each light source in a range of 0.1 to 1 W. In an embodiment, the reusable phototherapy module 700 includes a controller configured to independently drive first light sources at a first wavelength range and second light sources at a second wavelength range different from the first wavelength range. The controller selectively drives the first light sources and the second light sources according to a wound treatment protocol.

In an embodiment, the plurality of light sources 120 includes additional light source types beyond conventional LEDs and laser diodes. In an embodiment, the plurality of light sources 120 includes vertical-cavity surface-emitting lasers (VCSELs), which provide uniform beam profiles and are suitable for near-infrared applications due to their circular beam shape, low divergence, and ability to be arranged in high-density arrays. In an embodiment, the plurality of light sources 120 includes superluminescent diodes (SLDs), which provide broader spectral bandwidth than laser diodes while maintaining higher brightness than conventional LEDs, and exhibit lower coherence than lasers, which reduces speckle effects during phototherapy treatment. In an embodiment, the plurality of light sources 120 includes quantum dot LEDs (QLEDs), which provide tunable wavelength emission by varying the size of quantum dots, enabling precise selection of emission wavelengths for specific phototherapy applications. In an embodiment, the plurality of light sources 120 includes ultraviolet (UV) LEDs for sterilization and phototherapy applications. The UV LEDs emit electromagnetic radiation in UV-A wavelength ranges of 315-400 nm, UV-B wavelength ranges of 280-315 nm, or UV-C wavelength ranges of 100-280 nm. UV-C LEDs provide germicidal effects for wound sterilization, while UV-A and UV-B LEDs are used for specific dermatological phototherapy applications. In an embodiment, the plurality of light sources 120 includes edge-emitting laser diodes, which provide higher power output than VCSELs and are suitable for applications requiring deeper tissue penetration. In an embodiment, the plurality of light sources 120 includes phosphor-converted LEDs, wherein a blue or UV LED chip is combined with phosphor materials to produce light at different wavelengths, enabling a single LED package to emit multiple wavelengths or broadband white light. In an embodiment, the plurality of light sources 120 includes multi-junction LEDs, which stack multiple semiconductor layers to emit light at multiple wavelengths from a single chip. In an embodiment, the plurality of light sources 120 includes chip-on-board (COB) LED arrays, which provide high-density light output from a compact form factor by mounting multiple LED chips directly on a substrate without individual packaging.

In an embodiment, the wound treatment protocol includes a plurality of treatment modes. A first treatment mode provides a treatment duration of 60 seconds at an energy density of 6 J/cm² for superficial skin concerns and mild muscle relaxation. A second treatment mode provides a treatment duration of 120 seconds at an energy density of 12 J/cm² for moderate muscle soreness and joint discomfort. A third treatment mode provides a treatment duration of 300 seconds at an energy density of 30 J/cm² for deep tissue repair and chronic pain. A fourth treatment mode provides a treatment duration of 1200 seconds at an energy density of 60 J/cm² for comprehensive body treatment and post-surgical recovery. In an embodiment, treatment times extend up to 60 minutes. The controller allows the user adjustment of treatment time within a range of 0 to 60 minutes.

In an embodiment, the replaceable skin-contact patch 706 includes an identification element. The identification element is an RFID tag, an NFC tag, a resistor code, a QR code, or a conductive bridge. The reusable phototherapy module 700 is configured to detect the type or presence of the replaceable skin-contact patch 706 before activating the plurality of light sources 120. In an embodiment, the reusable phototherapy module 700 detects dressing type, dressing size, wound protocol, expiry date, whether the dressing is attached, or whether the dressing has been previously used. In an embodiment, the controller of the reusable phototherapy module 700 prevents operation when the replaceable skin-contact patch 706 has exceeded a permitted use count.

In an embodiment, the reusable phototherapy module 700 includes a controller that prevents activation of the plurality of light sources 120 when the replaceable skin-contact patch 706 is not coupled to the reusable phototherapy module 700. In an embodiment, the plurality of light sources 120 does not operate unless skin contact is detected, temperature is below a predetermined threshold, a treatment time is selected, or a battery level is sufficient. These safety interlock features help prevent unintended light exposure and improve the safety of the device.

In an embodiment, the detachable phototherapy device includes sensors for detecting wound parameters. The sensors include a temperature sensor for detecting wound temperature. The sensors further include sensors for detecting wound moisture and exudate levels, which provide reminders for dressing replacement. The sensors further include sensors for detecting bacterial presence through intelligent sensing. A controller adjusts the operation of the plurality of light sources 120 based on sensed wound parameters. In an embodiment, the controller provides zonal control of the plurality of light sources 120, enabling different light sources to be activated or deactivated independently based on the sensed wound parameters in different zones of the wound surface. In an embodiment, the controller provides wavelength mode control, enabling selection of different wavelength combinations based on the wound condition. In an embodiment, the device communicates with a mobile application for treatment monitoring and control. AI-assisted wound condition assessment is used to evaluate wound healing progress and select different phototherapy modes, forming a closed-loop mechanism of inspection, treatment, re-inspection, adjustment, and re-treatment.

In an embodiment, the controller includes or is communicatively coupled to an artificial intelligence (AI) processing module configured to provide AI-assisted wound assessment and treatment recommendations. The AI processing module receives input data from one or more sources, including wound images captured by an optional camera integrated into the reusable phototherapy module 700 or received from a mobile device via wireless communication, sensor data from the sensors, including temperature, moisture, and bacterial presence data, and treatment history data stored in a memory of the controller or received from a cloud-based server. The AI processing module implements one or more AI models, which include machine learning models, convolutional neural networks (CNNs) for image analysis, deep learning models, or rule-based expert systems. In an embodiment, the AI processing module performs wound assessment functions including wound size measurement, wound depth estimation, tissue classification such as identification of granulation tissue, necrotic tissue, and epithelial tissue, infection detection based on image analysis and sensor data, and healing stage classification. Based on the wound assessment, the AI processing module generates treatment recommendation outputs including phototherapy mode selection from among the plurality of treatment modes, wavelength selection for the first light sources and the second light sources, treatment duration, treatment frequency, and zonal control parameters for independently activating different light sources based on wound parameters in different zones. In an embodiment, the AI processing module includes learning and adaptation features, wherein the AI models are updated based on treatment outcomes to improve future recommendations. The AI processing module compares wound assessment data from successive treatment sessions to evaluate healing progress and adjusts treatment recommendations accordingly. In an embodiment, the AI processing module communicates with external devices, including mobile applications and cloud-based servers for remote monitoring, data storage, and cloud-based AI processing. Cloud-based processing enables access to more computationally intensive AI models and aggregated treatment outcome data from multiple devices. In an embodiment, data privacy considerations are addressed through encryption of wound images and treatment data, anonymization of data transmitted to cloud servers, and local processing options that do not require data transmission. The AI-assisted wound condition assessment forms part of a closed-loop treatment mechanism comprising inspection, treatment, re-inspection, adjustment, and re-treatment cycles.

In an embodiment, the replaceable skin-contact patch 706 includes a dynamically adaptive dressing layer having a multi-layer composite structure. An outer layer of the dynamically adaptive dressing layer is a directionally contractile thermally responsive hydrogel layer (DCTH). The DCTH layer is formed by poly(N-isopropylacrylamide) (PNIPAM) and sodium alginate (Alg) through a stretching-fixation process to form an anisotropic structure, followed by secondary cross-linking with Al³⁺. When triggered by 808 nm near-infrared light, the DCTH layer generates a directional contraction force of 20 to 30 kPa, which reduces mechanical tension at the wound edge. A middle layer of the dynamically adaptive dressing layer is a light-responsive antibacterial layer formed by cross-linking oxidized sodium alginate (OSA) and carboxymethyl chitosan (CMC), with embedded tannic acid-cerium (TA-Ce&sup4;+) photothermal nanozyme (PNZ) particles. Under near-infrared light irradiation, the PNZ particles simultaneously realize photothermal conversion and reactive oxygen species (ROS) scavenging, and synergize with LED phototherapy to inhibit bacterial proliferation, achieving an antibacterial rate of 99.8% or greater. An inner layer of the dynamically adaptive dressing layer is a medical-grade polyethylene glycol (PEG) hydrogel layer loaded with epidermal growth factor (EGF). At wound body surface temperatures of 32 to 37°C, the inner layer presents a gel state and seamlessly fits the wound surface to avoid secondary damage.

In an embodiment, three types of replaceable dressings are provided. A first type is a polyurethane perforated type with embedded hydrogel lens cylinders aligned with the light source positions. The polyurethane perforated type dressing includes growth-promoting factors, wound healing-promoting drugs, and an antibacterial coating 726. In an embodiment, the growth-promoting factors and wound healing-promoting drugs are applied as a surface coating on the hydrogel lenses or dressing surfaces. A second type is a full hydrogel type without perforations, serving as an optical conduction and wound contact layer with stretchable properties. When applied to a wound, the full hydrogel type exerts a wound-approximating effect similar to suturing. The full hydrogel type also includes growth-promoting factors and wound healing-promoting drugs, which are provided as a surface coating on the hydrogel surface. In an embodiment, optional antibacterial coatings are also applied to the full hydrogel type. A third type is an ultra-thin silicone type for superficial wounds, suitable for quick application.

In an embodiment, the detachable phototherapy device achieves an IPX4 to IPX6 waterproof rating, meeting clinical cleaning and humid environment requirements. The phototherapy light source module 704 adopts a potting and sealing process to achieve the waterproof rating.

In an embodiment, the reusable phototherapy module 700 includes a rechargeable lithium battery 126, for example, a 200 mAh battery, supporting Type-C fast charging. In an embodiment, the power source is detachable via a magnetic interface for separate charging. The reusable phototherapy module 700 further comprises a housing made of medical-grade ABS material, a controller, and a display for indicating treatment time and operational parameters. In an embodiment, the detachable phototherapy device has dimensions of approximately 60 x 60 mm.

In an embodiment, the detachable phototherapy device further includes a charging case or charging dock for the detachable power supply unit 702. The charging case is configured to receive the detachable power supply unit 702 for charging when the detachable power supply unit 702 is separated from the phototherapy light source module 704. The charging case includes a charging interface, such as a Type-C charging port, and includes a power indicator to display charging status. In an embodiment, the charging case is configured to fully charge the detachable power supply unit 702 in approximately 40 minutes.

In an embodiment, the detachable phototherapy device further includes a disposable protective component. The disposable protective component includes a sterile packaging sleeve and an adhesive fixing ring. The adhesive fixing ring uses a 3M low-sensitization pressure-sensitive adhesive. In an embodiment, pressure-sensitive strips are provided on an inner side of the adhesive fixing ring for real-time fitting feedback through the controller. When a fitting pressure falls below 15 kPa, the controller triggers an audible or visual alarm to alert the user.

In an embodiment, the replaceable skin-contact patch 706 is a disposable component, and the reusable phototherapy module 700 further comprises a housing, a controller, and a rechargeable battery 126. The reusable phototherapy module 700 is configured to be detached from the replaceable skin-contact patch 706 and coupled to a new replaceable skin-contact patch 706. The reusable phototherapy module 700 is reused for multiple treatment sessions while the replaceable skin-contact patch 706 is replaced after each use, thereby reducing waste and cost.

Several implementations have been described. Nevertheless, it will be understood that various modifications may be made without departing from the spirit and scope of the disclosure. Accordingly, other implementations are within the scope of the following claims.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A detachable phototherapy device, comprising:
a substrate layer comprising a first surface oriented towards a first direction, a second surface oriented towards a second direction opposite to the first direction;
a conductive layer printed on the first surface of the substrate layer, the conductive layer comprising a first conductor terminal and a second conductor terminal;
a plurality of light sources electrically coupled to the conductive layer, the plurality of light sources configured to emit electromagnetic radiation towards the first direction; and
a first electrode and a second electrode provided on the second surface, the first electrode electrically coupled to the first conductor terminal, and the second electrode electrically coupled to the second conductor terminal.

2. The detachable phototherapy device of claim 1, further comprising a battery having a first battery terminal and a second battery terminal, wherein in a use state, the battery is electrically coupled between the first electrode and the second electrode, the first battery terminal is electrically coupled to the first electrode and the second battery terminal is electrically coupled to the second electrode.

3. The detachable phototherapy device of claim 1, further comprising a replaceable skin-contact patch removably coupled to the substrate layer, the replaceable skin-contact patch including an adhesive skin-contact surface and at least one optically transmissive region aligned with the plurality of light sources.

4. The detachable phototherapy device of claim 3, wherein the at least one optically transmissive region comprises a plurality of concave-convex light-transmitting holes, each concave-convex light-transmitting hole having a first opening portion facing the substrate layer and a second opening portion facing a wound surface.

5. The detachable phototherapy device of claim 3, wherein the substrate layer and the replaceable skin-contact patch are coupled via magnetic attraction.

6. The detachable phototherapy device of claim 1, further comprising a deformable light board having a plurality of slots enabling multi-directional deformation, the substrate layer disposed on the deformable light board.

7. The detachable phototherapy device of claim 1, wherein the plurality of light sources includes first light sources configured to emit electromagnetic radiation in a red wavelength range and second light sources configured to emit electromagnetic radiation in a blue wavelength range.

8. A detachable phototherapy device comprising:
a reusable phototherapy module comprising a substrate layer having a first surface and a second surface, a conductive layer disposed on the first surface, a plurality of light sources electrically coupled to the conductive layer, and a power source;
a replaceable skin-contact patch removably coupled to the reusable phototherapy module, the replaceable skin-contact patch including a plurality of concave-convex light-transmitting holes aligned with the plurality of light sources, and
wherein each concave-convex light-transmitting hole includes a first opening portion facing the reusable phototherapy module and a second opening portion facing a wound surface.

9. The detachable phototherapy device of claim 8, wherein the reusable phototherapy module further comprises a first electrode and a second electrode disposed on the second surface of the substrate layer, the first electrode electrically coupled to a first conductor terminal of the conductive layer and the second electrode electrically coupled to a second conductor terminal of the conductive layer.

10. The detachable phototherapy device of claim 8, wherein the first opening portion is convex and the second opening portion is concave.

11. The detachable phototherapy device of claim 8, wherein an inner wall of each concave-convex light-transmitting hole is coated with an antibacterial coating.

12. The detachable phototherapy device of claim 8, wherein the replaceable skin-contact patch includes a skin-contact hydrogel layer and a polyurethane foam layer.

13. The detachable phototherapy device of claim 8, wherein the reusable phototherapy module includes a deformable light board having a plurality of slots enabling multi-directional deformation.

14. The detachable phototherapy device of claim 8, wherein the reusable phototherapy module includes a light-transmitting plate having a concave-convex lens structure, the light-transmitting plate and the plurality of concave-convex light-transmitting holes of the replaceable skin-contact patch forming a double-layer lens structure.

15. A detachable phototherapy device comprising:
a reusable phototherapy module comprising a substrate layer having a first surface and a second surface, a conductive layer disposed on the first surface, a plurality of light sources electrically coupled to the conductive layer, and a power source;
a replaceable skin-contact patch including an adhesive skin-contact surface and at least one optically transmissive region aligned with the plurality of light sources, and
wherein the reusable phototherapy module includes a plurality of first magnetic elements and the replaceable skin-contact patch includes a plurality of second magnetic elements, the plurality of first magnetic elements and the plurality of second magnetic elements configured to removably magnetically couple the reusable phototherapy module to the replaceable skin-contact patch.

16. The detachable phototherapy device of claim 15, wherein the reusable phototherapy module further comprises a first electrode and a second electrode disposed on the second surface of the substrate layer, the first electrode electrically coupled to a first conductor terminal of the conductive layer and the second electrode electrically coupled to a second conductor terminal of the conductive layer.

17. The detachable phototherapy device of claim 15, wherein the replaceable skin-contact patch includes an identification element, and wherein the reusable phototherapy module includes a controller configured to detect a type or presence of the replaceable skin-contact patch before activating the plurality of light sources.

18. The detachable phototherapy device of claim 17, wherein the controller prevents activation of the plurality of light sources when the replaceable skin-contact patch is not magnetically coupled to the reusable phototherapy module.

19. The detachable phototherapy device of claim 15, wherein the at least one optically transmissive region comprises a plurality of concave-convex light-transmitting holes, each concave-convex light-transmitting hole having a convex first opening portion and a concave second opening portion.

20. The detachable phototherapy device of claim 15, wherein the plurality of light sources includes first light sources and second light sources configured to emit electromagnetic radiation at different wavelengths, and wherein the reusable phototherapy module includes a controller configured to selectively drive the first light sources and the second light sources according to a wound treatment protocol.
